(19) United States
(12) Patent Application Publication
(10) Pub. No.: US 2023/0399626 A1
(43) Pub. Date: Dec. 14, 2023

(54) GENERATION OF CONDITIONED MEDIA FROM INDUCIBLE PLURIPOTENT STEM CELL DERIVED ENDOTHELIAL PROGENITOR CELLS

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Niceville, FL (US)

(73) Assignee: CREATIVE MEDICAL TECHNOLOGIES, INC., Phoenix, AZ (US)

(21) Appl. No.: 18/331,048

(22) Filed: Jun. 7, 2023

Related U.S. Application Data
(60) Provisional application No. 63/351,330, filed on Jun. 10, 2022.

(51) Int. Cl. C12N 5/071

(52) U.S. Cl. CPC C12N 5/0692; C12N 2501/60

(57) ABSTRACT

Disclosed are means, methods and compositions of matter useful for generation of conditioned media from endothelial progenitor cells (EPC). In one embodiment, EPC are extracted, dedifferentiated into inducible pluripotent stem cells (iPSC) and said iPSC are differentiated into the EPC lineage. The differentiated EPC are utilized as producers of conditioned media for therapeutic purposes. In one embodiment EPC are subjected to one or more stressors, after which conditioned media is extracted and in some cases concentrated. Said conditioned media can be utilized as a therapeutic agent or can be used in the generation of immune modulatory cells.

### CROSS-REFERENCE TO RELATED APPLICATIONS

This application claims priority to U.S. Provisional Application No. 63/351,330, titled “Generation of Conditioned Media from Inducible Pluripotent Stem Cell Derived Endothelial Progenitor Cells” filed Jun. 10, 2022, which is hereby incorporated by reference herein in its entirety.

### FIELD OF THE INVENTION

The invention relates to the generation of conditioned media from regenerative stem cell populations.

### BACKGROUND OF THE INVENTION

Stem cell therapies are slowly becoming a clinical reality. Initial thoughts in the area of regenerative medicine were that stem cells act as cells that give rise to new tissues. This concept, which held true for embryonic stem cells, was shown incorrect for adult stem cells. Generally speaking, when embryonic stem cells are administered into an area of injury, lets say a cardiac infarct, for example, the stem cells differentiate into cardiac tissues, and some left over stem cells do not differentiate and give rise to tumors called teratomas. Given the safety and ethical concerns of embryonic stem cells, clinical development has proceeded for adult stem cells.

Early studies showed that adult stem cells such as bone marrow, cord blood, and adipose stem cells can differentiate into other tissues. However, when male stem cells were administered into females, there was very little, if any, Y chromosome positive cells.

Currently the mechanisms that are believed to be responsible for the therapeutic effects of adult stem cells are believed to be a production of proteins and factors by the stem cells that activate endogenous repair processes. According to this line of thought, administration of stem cell derived factors, such as conditioned media, may be a promising approach to disease treatment. Unfortunately, there are several hurdles that inhibit the utilization of conditioned media as a medicine, one of them is that not enough stem cells or endothelial cells can be generated. The invention aims to overcome this.

### SUMMARY

Preferred embodiments include methods of creating an endothelial progenitor cell (EPC) conditioned media comprising the steps of: a) obtaining a cellular population; b) dedifferentiating said cellular population; c) inducing differentiation of said dedifferentiated cell into EPC; and d) culturing said EPC in a liquid media to obtain a conditioned media.

Preferred methods include embodiments wherein said cellular population is an EPC.

Preferred methods include embodiments wherein said EPC is derived from a source selected from the group consisting of: a) peripheral blood; b) bone marrow; c) placenta; d) cord blood; e) menstrual blood; f) amniotic fluid; and g) cerebral spinal fluid.

Preferred methods include embodiments wherein said EPC express CD33.

Preferred methods include embodiments wherein said EPC express CD34.

Preferred methods include embodiments wherein said EPC express CD9.

Preferred methods include embodiments wherein said EPC express CD133.

Preferred methods include embodiments wherein said EPC express VEGF-receptor 2.

Preferred methods include embodiments wherein said EPC express CD105.

Preferred methods include embodiments wherein said EPC express c-met.

Preferred methods include embodiments wherein said EPC express interleukin-3 receptor.

Preferred methods include embodiments wherein said EPC express IGF receptor.

Preferred methods include embodiments wherein said EPC express EGF receptor.

Preferred methods include embodiments wherein said EPC express LDL receptor.

Preferred methods include embodiments wherein said cellular population is monocytes.

Preferred methods include embodiments wherein said monocytes are type 2 monocytes.

Preferred methods include embodiments wherein said cellular population is thymic medullary epithelial cells.

Preferred methods include embodiments wherein said cellular population is hematopoietic stem cells.

Preferred methods include embodiments wherein said hematopoietic stem cells express CD34.

Preferred methods include embodiments wherein said hematopoietic stem cells express CD133.

Preferred methods include embodiments wherein said hematopoietic stem cells possess ability to generate lymphoid cells when administered into an immune deficient mouse.

Preferred methods include embodiments wherein said hematopoietic stem cells possess ability to generate myeloid cells when administered into an immune deficient mouse.

Preferred methods include embodiments wherein said hematopoietic stem cells possess ability to generate erythroid cells when administered into an immune deficient mouse.

Preferred methods include embodiments wherein said hematopoietic stem cells possess ability to generate megakaryocytic cells when administered into an immune deficient mouse.

Preferred methods include embodiments wherein said hematopoietic stem cells possess ability to generate megakaryocytic cells when administered into an immune deficient mouse.

Preferred methods include embodiments wherein said cellular population is a mesenchymal stem cell.

Preferred methods include embodiments wherein said mesenchymal stem cells are naturally occurring mesenchymal stem cells.

Preferred methods include embodiments wherein said mesenchymal stem cells are generated in vitro.

Preferred methods include embodiments wherein said naturally occurring mesenchymal stem cells are tissue derived.

Preferred methods include embodiments wherein said naturally occurring mesenchymal stem cells are derived from a bodily fluid.

Preferred methods include embodiments wherein said tissue derived mesenchymal stem cells are derived from the bone marrow.

Preferred methods include embodiments wherein said tissue derived mesenchymal stem cells are derived from perivascular tissue.

Preferred methods include embodiments wherein said tissue derived mesenchymal stem cells are derived from adipose tissue.

Preferred methods include embodiments wherein said tissue derived mesenchymal stem cells are derived from placental tissue.

Preferred methods include embodiments wherein said tissue derived mesenchymal stem cells are derived from amniotic tissue.

Preferred methods include embodiments wherein said tissue derived mesenchymal stem cells are derived from omental tissue.

Preferred methods include embodiments wherein said tissue derived mesenchymal stem cells are derived from deciduous tooth tissue.

Preferred methods include embodiments wherein said tissue derived mesenchymal stem cells are derived from umbilical cord tissue.

Preferred methods include embodiments wherein said tissue derived mesenchymal stem cells are derived from fallopian tube tissue.

Preferred methods include embodiments wherein said tissue derived mesenchymal stem cells are derived from hepatic tissue.

Preferred methods include embodiments wherein said tissue derived mesenchymal stem cells are derived from renal tissue.

Preferred methods include embodiments wherein said tissue derived mesenchymal stem cells are derived from cardiac tissue.

Preferred methods include embodiments wherein said tissue derived mesenchymal stem cells are derived from tonsillar tissue.

Preferred methods include embodiments wherein said tissue derived mesenchymal stem cells are derived from ovarian tissue.

Preferred methods include embodiments wherein said tissue derived mesenchymal stem cells are derived from neuronal tissue.

Preferred methods include embodiments wherein said tissue derived mesenchymal stem cells are derived from auricular tissue.

Preferred methods include embodiments wherein said tissue derived mesenchymal stem cells are derived from colonic tissue.

Preferred methods include embodiments wherein said tissue derived mesenchymal stem cells are derived from submucosal tissue.

Preferred methods include embodiments wherein said tissue derived mesenchymal stem cells are derived from hair follicle tissue.

Preferred methods include embodiments wherein said tissue derived mesenchymal stem cells are derived from hair pancreatic tissue.

Preferred methods include embodiments wherein said tissue derived mesenchymal stem cells are derived from hair muscle tissue.

Preferred methods include embodiments wherein said tissue derived mesenchymal stem cells are derived from hair subepithelial umbilical cord tissue.

Preferred methods include embodiments wherein said tissue derived mesenchymal stem cells are isolated from tissues containing cells selected from a group of cells comprising of: endothelial cells, epithelial cells, dermal cells, endodermal cells, mesodermal cells, stems, osteocytes, chondrocytes, natural killer cells, dendritic cells, hepatic cells, pancreatic cells, stromal cells, salivary gland mucous cells, and salivary gland serous cells.

Preferred methods include embodiments wherein said dedifferentiation is accomplished by introduction into cells proteins capable of inducing dedifferentiation.

Preferred methods include embodiments wherein said dedifferentiation results in cells expression pluripotency markers.

Preferred methods include embodiments wherein said pluripotency marker is TRA-1-60

Preferred methods include embodiments wherein said proteins capable of inducing dedifferentiation are selected from the group consisting of: a) OCT4; b) NANOG; c) KLF-1; d) SOX-2; and e) h-RAS.

Preferred methods include embodiments wherein mRNA is introduced into said cells in order to induce expression of pluripotency inducing genes.

Preferred methods include embodiments wherein said dedifferentiated cells are capable of proliferating for more than 50 passages.

Preferred methods include embodiments wherein said redifferentiated cells are capable of proliferating for more than 50 passages and have stable karyotype.

Preferred methods include embodiments wherein said redifferentiated cells are a direct therapeutic effector.

Preferred methods include embodiments wherein said redifferentiated cells are more potent than naturally occurring EPCs based on a) VEGF production b) nitric oxide release c) angiogenesis/capillary formation assay

### DETAILED DESCRIPTION OF THE INVENTION

The invention provides the generation of conditioned media by extracting cells, dedifferentiating cells, and using these dedifferentiated cells to create a stable and expandable population of endothelial progenitor cells (EPC). Said EPC can then be used for generation of conditioned media by culture of the EPC in the liquid media, or in some embodiments EPC are stimulated by activators and collection of induced proteins is performed.

As used herein and in the appended claims, the singular forms “a,” “and,” and “the” include plural referents unless the context clearly dictates otherwise. Thus, for example, reference to “a cell” includes a plurality of such cells and reference to “the agent” includes reference to one or more agents known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Although methods and materials similar or equivalent to those described herein can be used in the practice of the disclosed methods and compositions, the exemplary methods, devices and materials are described herein.

Unless defined otherwise, all technical and scientific terms used herein have the same meaning as commonly understood to one of ordinary skill in the art to which this disclosure belongs. Thus, as used throughout the instant application, the following terms shall have the following meanings.

While induced pluripotent stem cells (iPS cells) are virtually identical to ES cells at molecular and functional levels, there are critical hurdles to translation of their therapeutic potentials into medical applications. One of the issues is that because the current standard protocols for reprogramming and propagation of iPS cells include animal-derived materials that are unsuitable for potential clinical purposes, a fully defined method to generate and expand hiPS cells needs to be developed.

Induced pluripotent stem cells (iPS) are described by Shinya Yamanaka's team at Kyoto University, Japan. Yamanaka identified genes that are particularly active in embryonic stem cells, and used retroviruses to transfect mouse fibroblasts with a selection of those genes. Eventually, four key pluripotency genes essential for the production of pluripotent stem cells were isolated; Oct-3/4, SOX2, c-Myc, and Klf4. Cells were isolated by antibiotic selection for Fbx15+ cells. The same group published a study along with two other independent research groups from Harvard, MIT, and the University of California, Los Angeles, showing successful reprogramming of mouse fibroblasts into iPS and even producing a viable chimera.

The generation of human iPS cells by retroviral expression of four reprogramming factors (RFs; also referred to a de-differentiation factors) opened the potential for regenerative medicine therapies based on patient-specific, personalized stem cells. However, the insertional mutagenic potential of retroviruses combined with the potential for latent RF gene activation, especially c-MYC, all but eliminates integrative DNA-based approaches for use in regenerative medicine therapies. Other DNA-based iPS approaches using episomal vectors, adenovirus, integrated and excised piggyBac transposon or floxed lentivirus have been developed; however, these approaches either suffer from low efficiency of iPS cell generation or require genomic excision strategies that leaves behind an inserted DNA element tag. RNA-based iPS cell approaches using Sendai virus or mRNA transfection avoid potential integration problems associated with DNA-based approaches and are inherently safer methods for clinical applications. Although Sendai virus offers a reasonably efficient iPS approach, problems associated with persistent Sendai virus replication in iPS cell clones requires a negative selection step followed by several recloning steps from the single cell level to isolate virus-free iPS cells, such processes result in excessive iPS cellular division and passage. One of the more promising non-DNA based approaches involves daily transfection of four individual RF mRNAs (plus GFP mRNA) over 16 days. Unfortunately, this approach remains problematic. For example, experiments to replace KLF4 and retroviruses with corresponding transfected mRNAs were performed and the results validated; however, OCT4 and SOX2 retroviruses could not be replaced with transfected mRNAs. The problem appears to stem from both the rapid degradation of RF mRNAs combined with the inconsistent cell-to-cell threshold expression level variation over time, which derives from attempting to transfect four independent mRNAs into the same cell on a daily basis for >14 days during reprogramming. Consequently, there remains a significant need for a simple and highly reproducible, non-DNA based approach to generate human iPS cells.

The disclosure provides methods and compositions for generating iPS cells from somatic cells (e.g., fibroblast cells). The compositions and method comprise the use of replicons derived from alphaviruses. The replicons comprise an RNA sequence encoding non-structural alphavirus proteins necessary for replication and 1, 2, 3, 4 or more coding sequences heterologous to the alphavirus and which induce dedifferentiation of somatic cells to stem cell phenotypes.

As used herein, the term “alphavirus” has its conventional meaning in the art, and includes the various species such as Venezuelan Equine Encephalitis (VEE) Virus, Eastern Equine Encephalitis (EEE) virus, Everglades Virus (EVE), Mucambo Virus (MUC), Pixuna Virus (PIX), and Western Equine Encephalitis Virus, all of which are members of the VEE/EEE Group of alphaviruses. Other alphaviruses include, e.g., Semliki Forest Virus (SFV), Sindbis, Ross River Virus, Chikungunya Virus, S.A. AR86, Barmah Forest Virus, Middleburg Virus, O′nyong-nyong Virus, Getah Virus, Sagiyama Virus, Bebaru Virus, Mayaro Virus, Una Virus, Aura Virus, Whataroa Virus, Banbanki Virus, Kyzylagach Virus, Highlands J Virus, Fort Morgan Virus, Ndumu Virus, and Buggy Creek Virus. Alphaviruses particularly useful in the constructs and methods described herein are VEE/EEE group alphaviruses.

The terms “alphavirus RNA replicon”, “alphavirus replicon RNA”, “alphavirus RNA vector replicon”, and “vector replicon RNA” are used interchangeably to refer to an RNA molecule expressing nonstructural protein genes such that it can direct its own replication (amplification) and comprises, at a minimum, 5′ and 3′ alphavirus replication recognition sequences, coding sequences for alphavirus nonstructural proteins, and a polyadenylation tract. It may additionally contain one or more elements (e.g., IRES sequences, core or mini-promoters and the like) to direct the expression, meaning transcription and translation, of a heterologous RNA sequence. The alphavirus replicon of the disclosure can comprise, in one embodiment, 5′ and 3′ alphavirus replication recognition sequences, coding sequences for alphavirus nonstructural proteins, a polyadenylation tract and one or more of a coding sequence selected from the group consisting of SOX-2, c-Myc, OCT-3/4, Klf, Glis1 and Nanog.

The term “polynucleotide,” “nucleic acid” or “recombinant nucleic acid” refers to polynucleotides such as deoxyribonucleic acid (DNA), and, where appropriate (particularly with reference to a replicon), ribonucleic acid (RNA).

The term “expression” with respect to a gene or polynucleotide refers to transcription of the gene or polynucleotide and, as appropriate, translation of an mRNA transcript to a protein or polypeptide. Thus, as will be clear from the context, expression of a protein or polypeptide results from transcription and/or translation of the open reading frame.

The generation of pluripotent cells for the use in the current invention can be performed by various means known in the art. The invention teaches the process of reprogramming of EPC, or other cells to generate a pluripotent cell line, said cell line subsequently being differentiated to EPC, wherein said EPC are utilized for generation of conditioned media. The invention provides Sendai virus vectors incorporated with nuclear reprogramming factor (KLF, OCT, and SOX) genes in a specific order, and methods that use these vectors for delivering the reprogramming factor genes in the induction of reprogramming of differentiated cells, and in particular, methods for delivering the reprogramming factor genes in the production of pluripotent stem cells from somatic cells. Specifically, the methods comprise a step of contacting differentiated cells such as somatic cells with a Sendai virus vector incorporated with at least three genes encoding reprogramming factors, i.e., genes encoding KLF, OCT, and SOX in this order, or a Sendai virus vector incorporated with the OCT gene, SOX gene, and KLF gene in this order. More specifically, the present invention provides methods for delivering the reprogramming factor genes in cellular reprogramming, wherein the method comprises introducing the three reprogramming factor genes into cells in need thereof using the Sendai virus vector, and compositions comprising the Sendai virus vector for use in the method. In the present invention, “pluripotent stem cells” refer to stem cells produced from the inner cell mass of an embryo of an animal in the blastocyst stage or cells having phenotypes similar to those cells. Specifically, pluripotent stem cells induced in the present invention are cells that express alkaline phosphatase which is an indicator of ES-like cells. Furthermore, preferably, when pluripotent stem cells are cultured, they form flat colonies containing cells with a higher proportion of nucleus volume than cytoplasm. Culturing may be carried out suitably with a feeder. Moreover, while cultured cells such as MEF stop proliferating in a few weeks, pluripotent stem cells can be passaged for a long period of time, and this can be confirmed based on their proliferative character that is not lost even when they are passaged, for example, 15 times or more, preferably 20 times or more, 25 times or more, 30 times or more, 35 times or more, or 40 times or more every three days. Furthermore, pluripotent stem cells preferably express endogenous OCT3/4 or Nanog, or more preferably, they express both of them. Furthermore, pluripotent stem cells preferably express TERT, and show telomerase activity (activity to synthesize telomeric repeat sequences). Moreover, pluripotent stem cells preferably have the ability to differentiate into three germ layers (the endoderm, mesoderm, and ectoderm); for example, during teratoma formation and/or embryoid body formation. More preferably, pluripotent stem cells produce germline chimera when they are transplanted into blastocysts. Pluripotent stem cells capable of germline transmission are called germline-competent pluripotent stem cells. Confirmation of these phenotypes can be carried out by known methods (WO 2007/69666; Ichisaka T. et al., Nature 448 (7151): 313-7, 2007). Furthermore, in the present invention, “differentiated” refers to that a differentiation stage of a cell is progressed more than before, and may refers to, for example, be more differentiated as compared to pluripotent stem cells, and includes states still possessing the ability to differentiate into multiple cell lineages (for example, somatic stem cells) and terminally differentiated states. Differentiated cells are cells (other than pluripotent stem cells) derived from pluripotent stem cells. Differentiated cells may be, for example, cells that do not have the ability to differentiate into the three germ layers (the endoderm, mesoderm, and ectoderm). Such cells will not have the ability to form the three germ layers unless they are reprogrammed. Furthermore, differentiated cells may be, for example, cells that cannot produce cells that are not of the germ layer type to which they belong. Differentiated cells may be somatic cells, and for example, they may be cells other than germ cells.

In the present invention, reprogramming refers to converting the differentiation state of a particular cell to a less differentiated state, and includes for example, dedifferentiation of differentiated cells, such as inducing cells with differentiation pluripotency, for example pluripotent stem cells, from cells without differentiation pluripotency. Furthermore, in the present invention, dedifferentiation refers to converting a particular cell into a more premature (for example, undifferentiated) state. Dedifferentiation may be reversion of a cell to its initial state or intermediate state in its path of differentiation. Furthermore, dedifferentiation may be a change from a cell unable to produce cells that are not of the same germ layer type to which the cell belongs, into a cell that can differentiate into other germ layer type cells. Dedifferentiation also includes, for example, cells not having triploblastic differentiation ability acquiring this triploblastic differentiation ability. Additionally, dedifferentiation includes the production of pluripotent stem cells.

Furthermore, in the present invention, somatic cells are, for example, cells other than pluripotent stem cells and germ cells. Somatic cells include, for example, multicellular organism-constituting cells other than pluripotent stem cells, and cultured cells thereof. Somatic cells include for example, somatic stem cells and terminally differentiated cells.

In the present invention, virus vectors are vectors having genomic nucleic acids derived from the virus, and that can express transgenes by incorporating the transgenes into the nucleic acids. Since Sendai virus vectors are chromosomally non-integrating virus vectors and expressed in the cytoplasm, there is no risk that the introduced gene will become integrated into the chromosome (nucleus-derived chromosome) of the host. Therefore, the vectors are safe and can be removed after completion of reprogramming. In the present invention, Sendai virus vectors include infectious viral particles, as well as complexes of the viral core, viral genome, and viral proteins, and complexes comprising non-infectious viral particles and such, which are complexes having the ability to express loaded genes upon introduction into cells. For example, in Sendai viruses, ribonucleoproteins (the viral core portion) consisting of a Sendai virus genome and bound Sendai virus proteins (NP, P, and L proteins) can express transgenes in cells when they are introduced into cells (WO 00/70055). Introduction into cells can be appropriately carried out using transfection reagents and the like. Such ribonucleoproteins (RNPs) are also included in the Sendai virus vectors of the present invention.

Sendai virus is a Mononegavirales virus, belongs to Paramyxoviridae (including the genera Paramyxovirus, Morbillivirus, Rubulavirus, and Pneumovirus), and contains a single-stranded minus-strand (antisense strand of a viral protein-encoding sense strand) RNA as the genome. Minus-strand RNA is also called negative-strand RNA. Mononegavirales includes viruses belonging to families such as Rhabdoviridae (including the genera Vesiculovirus, Lyssavirus, and Ephemerovirus), and Filoviridae, in addition to Paramyxovirus (Paramyxoviridae virus) (Virus, vol. 57, No. 1: pp 29-36, 2007; Annu. Rev. Genet. 32, 123-162, 1998; Fields virology fourth edition, Philadelphia, Lippincott-Raven, 1305-1340, 2001; Microbiol. Immunol. 43, 613-624, 1999; Field Virology, Third edition pp. 1205-1241, 1996). Other examples of Paramyxoviridae virus other than Sendai virus include Newcastle disease virus, mumps virus, measles virus, respiratory syncytial virus (RS virus), rinderpest virus, distemper virus, simian parainfluenza virus (SV5), and human parainfluenza viruses I, II, and III; influenza virus belonging to the Orthomyxoviridae family; and the vesicular stomatitis virus and Rabies virus belonging to the Rhabdoviridae family. Further examples include Sendai virus (SeV), human parainfluenza virus-1 (HPIV-1), human parainfluenza virus-3 (HPIV-3), phocine distemper virus (PDV), canine distemper virus (CDV), dolphin molbillivirus (DMV), peste-des-petits-ruminants virus (PDPR), measles virus (MV), rinderpest virus (RPV), Hendra virus (Hendra), Nipah virus (Nipah), human parainfluenza virus-2 (HPIV-2), simian parainfluenza virus 5 (SV5), human parainfluenza virus-4a (HPIV-4a), human parainfluenza virus-4b (HPIV-4b), mumps virus (Mumps), and Newcastle disease virus (NDV). More preferably, examples include viruses selected from the group consisting of Sendai virus (SeV), human parainfluenza virus-1 (HPIV-1), human parainfluenza virus-3 (HPIV-3), phocine distemper virus (PDV), canine distemper virus (CDV), dolphin molbillivirus (DMV), peste-des-petits-ruminants virus (PDPR), measles virus (MV), rinderpest virus (RPV), Hendra virus (Hendra), and Nipah virus (Nipah).

For examples of accession numbers in the database for the nucleotide sequences of Sendai virus genes, see M29343, M30202, M30203, M30204, M51331, M55565, M69046, and X17218 for the NP gene; M30202, M30203, M30204, M55565, M69046, X00583, X17007, and X17008 for the P gene; D11446, K02742, M30202, M30203, M30204, M69046, U31956, X00584, and X53056 for the M gene; D00152, D11446, D17334, D17335, M30202, M30203, M30204, M69046, X00152, and X02131 for the F gene; D26475, M12397, M30202, M30203, M30204, M69046, X00586, X02808, and X56131 for the FIN gene; and D00053, M30202, M30203, M30204, M69040, X00587, and X58886 for the L gene. Examples of viral genes encoded by other viruses include CDV, AF014953; DMV, X75961; HPIV-1, D01070; HPIV-2, M55320; HPIV-3, D10025; Mapuera, X85128; Mumps, D86172; MV, K01711; NDV, AF064091; PDPR, X74443; PDV, X75717; RPV, X68311; SeV, X00087; SV5, M81442; and Tupaia, AF079780 for the NP gene (also referred to as the N gene); CDV, X51869; DMV, Z47758; HPIV-1, M74081; HPIV-3, X04721; HPIV-4a, M55975; HPIV-4b, M55976; Mumps, D86173; MV, M89920; NDV, M20302; PDV, X75960; RPV, X68311; SeV, M30202; SV5, AF052755; and Tupaia, AF079780 for the P gene; CDV, AF014953; DMV, Z47758; HPIV-1. M74081; HPIV-3, D00047; MV, AB016162; RPV, X68311; SeV, AB005796; and Tupaia, AF079780 for the C gene; CDV, M12669; DMV 230087; HPIV-1, 538067; HPIV-2, M62734; HPIV-3, D00130; HPIV-4a, D10241; HPIV-4b, D10242; Mumps, D86171; MV, AB012948; NDV, AF089819; PDPR, Z47977; PDV, X75717; RPV, M34018; SeV, U31956; and SV5, M32248 for the M gene; CDV, M21849; DMV, AJ224704; HPN-1, M22347; HPIV-2, M60182; HPIV-3. X05303, HPIV-4a, D49821; HPIV-4b, D49822; Mumps, D86169; MV, AB003178; NDV, AF048763; PDPR, Z37017; PDV, AJ224706; RPV, M21514; SeV, D17334; and SV5, AB021962 for the F gene; and CDV, AF112189; DMV, AJ224705; HPIV-1, U709498; HPIV-2. D000865; HPIV-3, AB012132; HPIV-4A, M34033; HPIV-4B, AB006954; Mumps, X99040; MV, K01711; NDV, AF204872; PDPR, Z81358; PDV, Z36979; RPV, AF132934; SeV, U06433; and SV-5, 576876 for the HN(H or G) gene. However, multiple strains are known for each of the viruses, and genes consisting of a sequence other than those exemplified above may exist due to strain differences. Sendai virus vectors carrying viral genes derived from any of these genes are useful as vectors of the present invention. For example, Sendai virus vectors of the present invention comprise a nucleotide sequence having 90% or higher, preferably 95% or higher, 96% or higher, 97% or higher, 98% or higher, or 99% or higher identity to the coding sequence of any of the above-mentioned viral genes. Furthermore, the Sendai virus vectors of the present invention comprise, for example, a nucleotide sequence encoding an amino acid sequence having 90% or higher, preferably 95% or higher, 96% or higher, 97% or higher, 98% or higher, or 99% or higher identity to an amino acid sequence encoded by the coding sequence of any one of the above-mentioned viral genes. Furthermore, Sendai virus vectors of the present invention comprise, for example, a nucleotide sequence encoding an amino acid sequence with ten or less, preferably nine or less, eight or less, seven or less, six or less, five or less, four or less, three or less, two or less, or one amino acid substitutions, insertions, deletions, and/or additions in an amino acid sequence encoded by the coding sequence of any one of the above-mentioned viral genes.

The sequences referenced by the database accession numbers such as the nucleotide sequences and amino acid sequences described herein refer to sequences on, for example, the filing date and priority date of this application, and can be identified as sequences at the time of either the filing date or priority date of the present application, and are preferably identified as sequences on the filing date of this application. The sequences at the respective time points can be identified by referring to the revision history of the database.

The Sendai virus vectors used in the present invention may be derivatives, and the derivatives comprise viruses with modified viral genes, and chemically modified viruses, and such, so that the ability of the virus to introduce genes is not impaired.

Furthermore, Sendai viruses may be derived from natural strains, wild-type strains, mutant strains, laboratory-passaged strains, and artificially constructed strains and such. An example is the Z strain (Medical Journal of Osaka University Vol. 6, No. 1, March 1955 p 1-15). That is, these viruses may be virus vectors having similar structures as viruses isolated from nature, or viruses artificially modified by genetic recombination, as long as the desired reprogramming can be induced. For example, they may have mutations or deletions in any of the genes of the wild-type virus. Furthermore, incomplete viruses such as DI particles (J. Virol. 68: 8413-8417, 1994) may also be used. For example, viruses having a mutation or deletion in at least one gene encoding a viral envelope protein or a coat protein can be preferably used. Such virus vectors are, for example, virus vectors that can replicate the genome in infected cells but cannot form infectious virus particles. Since there is no worry of spreading the infection to the surroundings, such transmission-defective virus vectors are highly safe. For example, minus-strand RNA viruses that do not contain at least one gene encoding an envelope protein such as F and/or HN or a spike protein, or a combination thereof may be used (WO 00/70055 and WO 00/70070; Li, H.-O. et al., J. Virol. 74(14): 6564-6569 (2000)). If proteins necessary for genome replication (for example, N, P, and L proteins) are encoded in the genomic RNA, the genome can be amplified in infected cells. To produce defective type of viruses, for example, the defective gene product or a protein that can complement it is externally supplied in the virus-producing cell (WO 00/70055 and WO 00/70070; Li, H.-O. et al., J. Virol. 74(14): 6564-6569 (2000)). Furthermore, a method of collecting virus vectors as noninfective virus particles (VLP) without completely complementing the defective viral protein is also known (WO 00/70070). Furthermore, when virus vectors are collected as RNPs (for example, RNPs containing the N, L, and P proteins and genomic RNA), vectors can be produced without complementing the envelope proteins.

Furthermore, the use of virus vectors carrying a mutant viral protein gene is also preferred. The present invention particularly provides methods of gene delivery in reprogramming, methods for producing reprogrammed cells, compositions, and kits using Sendai virus vectors having mutations and/or deletions in the viral gene. For example, in the envelope protein and coat proteins, many mutations including attenuation mutations and temperature-sensitive mutations are known. Sendai viruses having these mutant protein genes can be used favorably in the present invention. In the present invention, vectors with lowered cytotoxicity are desirably used. Cytotoxicity can be measured, for example by quantifying the release of lactic acid dehydrogenase (LDH) from cells. For example, vectors with significantly lowered cytotoxicity compared to the wild type can be used. Regarding the degree of lowering of cytotoxicity, for example, vectors showing a significant decrease of, for example 20% or more, 25% or more, 30% or more, 35% or more, 40% or more, or 50% or more in the LDH release level compared to the wild-type in a culture medium of human-derived HeLa cell (ATCC CCL-2) or simian-derived CV-1 cell (ATCC CCL 70) infected at MOI (multiplicity of infection) 3 and cultured for three days can be used. Furthermore, mutations that decrease cytotoxicity also include temperature-sensitive mutations. Temperature-sensitive mutations refer to mutations which significantly decrease the activity at the viral host's ordinary temperature (for example, 37° C. to 38° C.) when compared to that at a low temperature (for example, 30° C. to 32° C.). Such proteins with temperature-sensitive mutations are useful since the viruses can be produced under permissive temperatures (low temperatures). When infected at 37° C., the virus vectors having useful temperature-sensitive mutations in the present invention show, a growth rate or gene expression level of, for example, ½ or less, preferably ¼ or less, more preferably ⅓ or less, more preferably 1/10 or less, and more preferably 1/20 or less compared to when cultured cells are infected at 32° C.

A Sendai virus vector used in the present invention may be a wild type as long as it does not inhibit reprogramming and can induce reprogramming by reprogramming factors or support induction of reprogramming, and has deletions or mutations in preferably at least one, more preferably at least 2, 3, 4, 5, or more viral genes. Deletions and mutations may be arbitrarily combined and introduced to each of the genes. Herein, a mutation may be a function-impairing mutation or a temperature-sensitive mutation, and is a mutation that decreases the viral proliferation rate or the expression level of any of the carried gene to preferably ½ or less, more preferably ¼ or less, more preferably ⅓ or less, more preferably 1/10 or less, and more preferably 1/20 or less compared to the wild type at least at 37° C. The use of such modified virus vectors can be useful particularly for the induction of pluripotent stem cells. For example, Sendai virus vectors used favorably in the present invention have at least two deleted or mutated viral genes. Such viruses include those with deletions of at least two viral genes, those with mutations in at least two viral genes, and those with a mutation in at least one viral gene and a deletion of at least one viral gene. The at least two mutated or deleted viral genes are preferably genes encoding envelope-constituting proteins. For example, vectors with deletion of the F gene with further deletion of the M and/or the HN gene or further mutation (for example, temperature-sensitive mutation) in the M and/or the FIN gene are used favorably in the present invention. Furthermore, for example, vectors with deletion of the F gene with further deletion of the M or the HN gene and further mutation in the remaining M and/or HN gene (for example, temperature-sensitive mutation) are also used favorably in the present invention. Vectors used in the present invention more preferably have at least three deleted or mutated viral genes (preferably at least three genes encoding envelope-constituting proteins; F, HN, and M). Such virus vectors include those with deletion of at least three genes, those with mutations in at least three genes, those with mutations in at least one gene and deletion of at least two genes, and those with mutations in at least two genes and deletion of at least one gene. As examples of more preferred embodiments, vectors with deletion of the F gene with further deletion of the M and the FIN gene or further mutations (for example, temperature-sensitive mutations) in the M and the HN gene are used favorably in the present invention. Furthermore, for example, vectors with deletion of the F gene with further deletion of the M or the FIN gene and further mutation in the remaining M or HN gene (for example, temperature-sensitive mutation) are used favorably in the present invention. Such mutated-forms of viruses can be produced according to known methods. For example, a temperature-sensitive mutation of the M gene of Sendai virus includes amino acid substitution of a site arbitrarily selected from the group consisting of position 69 (G69), position 116 (T116), and position 183 (A183) of the M protein (Inoue, M. et al., J. Virol. 2003, 77: 3238-3246). Viruses having a genome encoding a mutant M protein, in which the amino acids of any one site, preferably a combination of any two sites, or more preferably all three sites of the three sites mentioned above are substituted in the Sendai virus M proteins to other amino acids, are used preferably in the present invention.

Preferred amino acid mutations are substitution to other amino acids with a side chain having different chemical properties, and examples are substitution to an amino acid with a BLOSUM62 matrix (Henikoff, S, and Henikoff, J. G. (1992) Proc. Natl. Acad. Sci. USA 89: 10915-10919) score of three or less, preferably two or less, more preferably one or less, and even more preferably 0. Specifically, G69, T116, and A 183 of the Sendai virus M protein can be substituted to Glu (E), Ala (A), and Ser (S), respectively. Alternatively, mutations homologous to mutations in the M protein of the temperature-sensitive P253-505 measles virus strain (Morikawa, Y. et al., Kitasato Arch. Exp. Med. 1991, 64: 15-30) can also be used. Mutations can be introduced according to known mutation methods, for example, using oligonucleotides and such.

Furthermore, examples of temperature-sensitive mutations in the HN gene include amino acid substitution of a site arbitrarily selected from the group consisting of position 262 (A262), position 264 (G264), and position 461 (K461) of the HN protein of a Sendai virus (Inoue, M. et al., J. Virol. 2003, 77: 3238-3246). Viruses having a genome encoding a mutant HN protein in which the amino acids of any one of the three sites, preferably a combination of any two sites, or more preferably all three sites are substituted to other amino acids are used preferably in the present invention. As mentioned above, preferred amino acid substitutions are substitutions to other amino acids with a side chain having different chemical properties. As a preferred example, A262, G264, and K461 of the Sendai virus HN protein are substituted to Thr (T), Arg (R), and Gly (G), respectively. Furthermore, for example, using the temperature-sensitive vaccine strain Urabe AM9 of the mumps virus as a reference, amino acids at positions 464 and 468 of the HN protein can be mutated (Wright, K. E. et al., Virus Res. 2000, 67: 49-57).

Furthermore, Sendai viruses may have mutations in the P gene and/or the L gene. Examples of such mutations are specifically, mutation of Glu at position 86 (E86) in the SeV P protein, and substitution of Leu at position 511 (L511) in the SeV P protein to other amino acids. As mentioned above, preferred amino acid substitutions are substitutions to other amino acids with a side chain having different chemical properties. Specific examples include substitution of the amino acid at position 86 to Lys, and substitution of the amino acid at position 511 to Phe. Furthermore, examples in the L protein include substitution of Asn at position 1197 (N1197) and/or Lys at position 1795 (K1795) in the SeV L protein to other amino acids, and similarly as above, preferred amino acid substitutions are substitutions to other amino acids with a side chain having different chemical properties. Specific examples are substitution of the amino acid at position 1197 to Ser, and substitution of the amino acid at position 1795 to Glu. Mutations of the P gene and L gene can significantly increase the effects of sustained infectivity, suppression of release of secondary particles, or suppression of cytotoxicity. Further, combination of mutations and/or deletions of envelope protein genes can dramatically increase these effects. Furthermore, examples for the L gene include substitution of Tyr at position 1214 (Y1214) and/or substitution of Met at position 1602 (M1602) of the SeV L protein to other amino acids, and similarly as above, preferred amino acid substitutions are substitutions to other amino acids with a side chain having different chemical properties. Specific examples are substitution of the amino acid at position 1214 to Phe, and substitution of the amino acid at position 1602 to Leu. The above-mentioned mutations can be arbitrarily combined.

For example, Sendai virus vectors in which at least G at position 69, T at position 116, and A at position 183 of the SeV M protein, at least A of position 262, G of position 264, and K of position 461 of the SeV HN protein, at least L of position 511 of the SeV P protein, and at least N of position 1197 and K of position 1795 of the SeV L protein are each substituted to other amino acids, and in which the F gene is also deficient or deleted; and F-gene-deleted or -deficient Sendai virus vectors whose cytotoxicity is similar to or lower than those mentioned above and/or whose temperature sensitivity is similar to or higher than those mentioned above are particularly preferred for the expression of nuclear reprogramming factors in the present invention. Specific examples of the substitutions include G69E, T116A, and A183S substitutions for the M protein, A262T, G264R, and K461G substitutions for the HN protein, L511F substitution for the P protein, and N1197S and K1795E substitutions for the L protein.

Examples of mutations of the L protein include substitutions of an amino acids at sites arbitrarily selected from position 942 (Y942), position 1361 (L1361), and position 1558 (L1558) of the SeV L protein to other amino acids. Similarly, as above, preferred amino acid substitutions are substitutions to other amino acids with a side chain having different chemical properties. Specific examples include substitution of the amino acid at position 942 to H is, substitution of the amino acid at position 1361 to Cys, and substitution of the amino acid at position 1558 to Ile. In particular, the L protein with substitutions at least at positions 942 or 1558 can be used preferably. For example, mutant L proteins in which, in addition to position 1558, position 1361 is also substituted to another amino acid are preferred as well. Furthermore, mutant L proteins in which, in addition to position 942, position 1558 and/or position 1361 are also substituted to other amino acids are favorable as well. These mutations can increase the temperature sensitivity of the L protein.

Examples of mutations of the P protein include substitutions of amino acids at sites arbitrarily selected from position 433 (D433), position 434 (R434), and position 437 (K437) of the SeV P protein to other amino acids. Similarly, as above, preferred amino acid substitutions are substitutions to other amino acids with a side chain having different chemical properties. Specific examples include substitution of the amino acid at position 433 to Ala (A), substitution of the amino acid at position 434 to Ala (A), and substitution of the amino acid at position 437 to Ala (A). In particular, P proteins in which all three of these sites are substituted can be used preferably. These mutations can increase the temperature sensitivity of the P protein.

F-gene-deleted or -deficient Sendai virus vectors encoding a mutant P protein in which at least at the three positions of D at position 433, R at position 434, and K at position 437 of the SeV P protein are substituted to other amino acids, and a mutant L protein in which at least the L at position 1558 of the SeV L protein is substituted (preferably a mutant L protein in which at least the L at position 1361 is also substituted to another amino acid); and F-gene-deleted or -deficient Sendai virus vectors whose cytotoxicity is similar to or lower than those mentioned above and/or whose temperature sensitivity is similar to or higher than those mentioned above are used preferably in the present invention. In addition to the above-mentioned mutations, each of the viral proteins may have mutations on other amino acids (for example, on ten or less, five or less, four or less, three or less, two or less, or one amino acid). Since vectors comprising the above-mentioned mutations show a high temperature sensitivity, after completion of reprogramming, the vectors can be removed easily by culturing the cells at a slightly high temperature (for example, 37.5° C. to 39° C. preferably 38° C. to 39° C., or 38.5° C. to 39° C.).

An aspect of the present invention relates to a method of differentiating pluripotent cells into hematopoietic precursor cells or endothelial cells comprising the sequential steps of: (a) culturing or maintaining a plurality of substantially undifferentiated pluripotent cells in a first defined media comprising at least one growth factor, (b) incubating the cells in a second defined media which is essentially free of BMP4, VEGF, IL-3, Flt3 ligand, and GMCSF, (c) culturing the cells in a third defined media comprising an amount of BMP4 and VEGF sufficient to expand or promote differentiation in a plurality of the cells, and (d) culturing the cells in a fourth defined media comprising an amount of either (1) IL-3 and Flt3 ligand, or (2) VEGF, FGF-2 or an FGF-2 mimic, and IGF sufficient to expand or promote differentiation in a plurality of the cells; wherein a plurality of the pluripotent cells are differentiated into hematopoietic precursor cells or endothelial cells. In certain embodiments, combination (1) above may be used to promote differentiation into hematopoietic precursor cells. Combination (2) above may be used to promote differentiation into an endothelial cell or an endothelial progenitor cell. The second defined media may be free or essentially free of FGF-2, IL6, SCF and/or TPO. The third defined media may also include FGF-2 (e.g., from about 5-50 ng/ml or from about 10-25 ng/ml) or an FGF-2 mimic. As shown in the below examples, inclusion of FGF-2 in the third media can increase the efficiency of differentiation of pluripotent cells into hematopoietic precursor cells. In certain embodiments, the fourth defined media further comprises GMCSF, or at least one of IL-6, SCF, or TPO. In certain embodiments, the fourth defined media includes an amount of either: (1) IL-3, Flt3 ligand, and GMCSF, or (2) IL-3, Flt3 ligand, SCF, IL-6, and TPO sufficient to promote differentiation of the cells. The third defined media and/or the fourth defined media may further comprise BIT9500 or Serum Replacement 3. The method may comprise culturing cells in a defined media which includes BIT9500 or Serum Replacement 3. At least some of the cells may be at least partially separated or are substantially individualized prior to step (b). The cells may be substantially individualized using an enzyme, such as a trypsin. The cells may be contacted with a ROCK inhibitor and a trypsin inhibitor (e.g., a soybean trypsin inhibitor) subsequent to said individualization. The ROCK inhibitor may be selected from the list consisting of HA-100, H-1152, and Y-27632. A plurality of the pluripotent cells may form embryoid bodies (EBs). From about 200 to about 1000 cells per aggregate may be used to generate at least one of said EBs. The method may comprise culturing the cells at an atmospheric pressure of less than 20% oxygen or at an atmospheric pressure of about 5% oxygen. As shown in the below examples, differentiating cells under hypoxic conditions, such as at about 5% atmospheric O.sub.2, can increase the differentiation of the cells, e.g., into EPC.

In certain embodiments, said cells may be partially or substantially reaggregated at least once. The cells may be reaggregated after culture in the third defined media and prior to or during culture in the fourth defined media. The reaggregation may comprise exposing said cells to trypsin or TRYPLE. Said cells may be exposed to a ROCK inhibitor subsequent to the reaggregation, or said cells may be cultured in a media essentially free of a ROCK inhibitor subsequent to the reaggregation. The method may further comprise culturing the cells at an atmospheric pressure of less than about 20% oxygen, wherein from about 200 to about 1000 cells per aggregate are used to generate a plurality of embryoid bodies (EBs). The first defined media may comprise TeSR, mTeSR, or mTeSR1. Step (a) may comprise culturing the cells on a matrix-coated surface. The matrix may comprise laminin, vitronectin, gelatin, polylysine, thrombospondin or Matrigel™. The second defined media may comprise TeSR-GF or X-vivo15 media. The second defined media may further comprise about 0.1 ng/ml TGF-.beta. and about 20 ng/ml FGF-2. Step (b) may comprise incubating the cells for a period of from about 12 hours to about 3 days. Step (c) may comprise culturing or differentiating the cells for a period of from about 4 to about 8 days. Step (d) may comprise culturing the cells for a period of at least about 4, or from about 4 to about 8 days. A plurality of the pluripotent cells may be differentiated into EPC In certain embodiments, the EPC co-express CD31, CD43, and CD45. The myeloid progenitor cells may be common myeloid progenitors. The third defined media comprises about 10-50 ng/ml BMP4 and about 10-50 ng/ml VEGF. In certain embodiments, the third defined media further comprises 10-50 ng/ml FGF-2. The third defined media comprises about 25 ng/ml BMP4 and about 25 ng/ml VEGF. The fourth defined media may comprise about 5-25 ng/ml IL-3 and about 10-50 ng/ml Flt3 ligand. The fourth defined media may further comprise about 5-25 ng/ml GMCSF, or about 10-100 ng/ml or about 10-50 ng/ml TPO, about 10-100 ng/ml SCF, about 5-25 ng/ml IL-6, and about 5-25 ng/ml IL-3. The fourth defined media may comprise about 10 ng/ml IL-3, about 25 ng/ml Flt3 ligand, and about 10 ng/ml GMCSF. A plurality of the hematopoietic precursor cells may express at least two cell markers selected from the list comprising CD43, CD34, CD31 and CD45. A plurality of the hematopoietic precursor cells may express CD34, CD43, CD45 and CD31. In certain embodiments, the hematopoietic precursor cells are multipotent hematopoietic precursor cells that co-express CD34, CD43, CD45 and CD31. In certain embodiments, a fifth defined media may be used to further promote differentiation of the cells into a particular cell type; for example, various media may be used to promote differentiation of the hematopoietic precursor cells into a more differentiated cell type such as, for example, an erythroblast, a NK cell, or a T cell. The method may further comprise culturing a plurality of said cells in a fifth defined media comprising one or more growth factor selected from the list consisting of IL-3, IL-6, SCF, EPO, and TPO, in an amount sufficient to promote differentiation of a plurality of the cells into erythroblasts. A plurality of the cells are cultured in a fifth defined media comprising one or more growth factor selected from the list consisting of IL-7, SCF, and IL-2, in an amount sufficient to promote differentiation of the cells into NK cells. The method may further comprise culturing a plurality of said cells in a fifth defined media comprising Notch ligand and one or more growth factor selected from the list consisting of IL-7, SCF, and IL-2 in an amount sufficient to promote differentiation of the cells into T cells. The Notch ligand may be the Fc chimeric Notch DLL-1 ligand or Notch ligand produced by a stromal cell line which over-expresses the Notch ligand. In certain embodiments, a thymic peptide such thymosin alpha, thymopenin, or thymosin B4 may be used to further promote differentiation of the cells into T cells. In certain embodiments, the plurality of said cells comprise hematopoietic precursor cells. The third defined media may comprise one or more growth factor selected from the list consisting of SCF, IL-6, G-CSF, EPO, TPO, FGF2, IL-7, IL-11, IL-9, IL-13, IL-2, or M-CSF in an amount sufficient to promote expansion or further differentiation of the cells. The fourth defined media may comprise one or more growth factor selected from the list consisting of SCF, IL-6, G-CSF, EPO, TPO, FGF2, BMP4, VEGF, IL-7, IL-11, IL-9, IL-13, IL-2, or M-CSF in an amount sufficient to promote expansion or further differentiation of the cells. In certain embodiments, the method may comprise incubating the cells in a fifth defined media which includes one or more growth factor selected from the list consisting of SCF, IL-6, G-CSF, EPO, TPO, FGF2, IL-7, IL-11, IL-9, IL-13, IL-2, or M-CSF in an amount sufficient to promote expansion or further differentiation of the cells. Said pluripotent cells are preferably mammalian pluripotent cells. In certain embodiments the pluripotent cells are human pluripotent cells, such as human embryonic stem cells (hESC) or induced pluripotent cells (iPSC). The hESC comprise cells may be selected from the list consisting of H1, H9, hES2, hES3, hES4, hES5, hES6, BG01, BG02, BG03, HSF1, HSF6, H1, H7, H9, H13B, and H14. Said iPSC may be selected from the list consisting of iPS6.1, iPS 6.6, iPS, iPS 5.6, iPS iPS 5.12, iPS 5.2.15, iPS iPS 5.2.24, iPS 5.2.20, iPS 6.2.1, iPS-B1-SONL, iPS-B1-SOCK, iPS-TIPS lEE, iPS-TiPS IB, iPS-KIPS-5, and iPS 5/3-4.3.

In one embodiment, the invention teaches the generation of culture media obtained from EPC generated from dedifferentiated cells. For use within the invention, EPCs in the culture are in a proliferative state, said proliferative state being described as the cells not reaching confluency. In an ideal condition, said EPC cells are growing in a 25%-75% confluent state.

Stimulation of EPC production of regenerative factors is disclosed through the treatment of EPC with various agents that replicate injury associated conditions. In particular, injury associated conditions that possess necrotic or necroptosis characteristics. In one embodiment EPC are treated with HMGB1 or peptides derived thereof. Treatment of EPC with HMGB1 utilized in the context of the invention to induce production of various growth factors including EGF, FGF-1, FGF-2, FGF-5, FGF-17 and FGF-18. Additionally, upregulation of regenerative factor production is further disclosed in the invention by combination of HMGB1 administration together with agonists of the toll like receptor family such as the toll like receptor 2 agonist PAM3 CSK4, the toll like receptor 4 agonist lipopolysaccharide, and the toll like receptor 9 agonist, CpG. TLRs can also bind with damage-associated molecular patterns (DAMP) produced under stress or by tissue damage or cell apoptosis. It is believed that TLRs build a bridge between innate immunity and autoimmunity. There are five adaptors to TLRs including MyD88, TRIF, TIRAP/MAL, TRAM, and SARM. Upon activation, TLRs recruit specific adaptors to initiate the downstream signaling pathways leading to the production of inflammatory cytokines and chemokines. Under certain circumstances, ligation of TLRs drives to aberrant activation and unrestricted inflammatory responses, thereby contributing to the perpetuation of inflammation in autoimmune diseases. In the past, most studies focused on the intracellular TLRs, such as TLR3, TLR7, and TLR9, but recent studies reveal that cell surface TLRs, especially TLR2 and TLR4, also play an essential role in the development of autoimmune diseases and afford multiple therapeutic targets.

In some embodiments of the invention EPC may be expanded and cultured in a growth media in order to obtain conditioned media. The term Growth Medium generally refers to a medium sufficient for the culturing of EPC. In particular, one presently preferred medium for the culturing of the cells of the invention herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases, different growth media are used, or different supplementations are provided, and these are normally indicated in the text as supplementations to Growth Medium. Also relating to the present invention, the term standard growth conditions, as used herein refers to culturing of cells at 37.degree. C., in a standard atmosphere comprising 5% CO.sub.2. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO.sub.2, relative humidity, oxygen, growth medium, and the like.

Methods are provided wherein EPC that are used in the invention can undergo at least 25, 50, 100, or 1000 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.16, or 10.sup.17 or more cells when seeded at from about 10.sup.3 to about cells/cm.sup.2 in culture. Preferably these cell numbers are produced within 80, 70, or days or less. In one embodiment, EPC used for the generation of conditioned media are isolated and expanded, and possess one or more markers selected from the group consisting of CD10, CD13, CD44, CD73, CD90, CD141, PDGFr-alpha, or HLA-A, -B, -C. In addition, the cells do not produce one or more of CD31, CD34, CD45, CD117, CD141, or HLA-DR, DP, DQ.

For isolating a population of EPCs, the method comprising providing a cell culture derived from dedifferentiated cells and enriching for a population of cells that are about 6-12 micrometers in size, wherein the EPC express at least one of Oct-4, Nanog, Sox-2, KLF4, c-Myc, Rex-1, GDF-3, LIF receptor, CD105, CD117, CD344 and Stella, and does not express at least one of MHC class I, MHC class II, CD45, CD13, CD49c, CD66b, CD73, CD105, or CD90 cell surface proteins.

In some embodiments, the method optionally includes the step of depleting cells from the population expressing stem cell surface markers or MHC proteins, thereby isolating a population of stem cells. In some aspects, the cells to be depleted express MHC class I, CD66b, glycophorin a, or glycophorin b. In some aspects, the method optionally includes transfecting the cells with a polynucleotide vector containing a stem cell-specific promoter operably linked to a reporter or selection gene. In some aspects, the cell-specific promoter is an Oct-4, Nanog, Sox-9, GDF3, Rex-1, or Sox-2 promoter. In some embodiments, the method further includes the step of enriching the population for the EPCs using expression of a reporter or selection gene. In some embodiments, the method further includes the step of enriching the population of the EPCs by flow cytometry. In some embodiments, the method further includes the steps of contacting the cells with a detectable compound that enters the cells, the compound being selectively detectable in proliferating and non-proliferating cells; and enriching the population of cells for the proliferating cells. In some aspects, the detectable compound is carboxyfluorescein diacetate, succinimidyl ester, or Aldefluor. In some embodiments, method further includes culturing the cells under conditions that form tissue aggregate bodies. In some embodiments, the method further includes culturing the population of EPC under conditions that support proliferation of the cells.

In some embodiments, the method further includes separating cell types such as granulocytes, T-cells, B-cells, NK-cell, red blood cells, or any combination thereof, from the EPC. In some aspects, separating the cell types is done by cell depletion. Further embodiments of the current disclosure relate to a method of identifying a EPC1, the method comprises the steps of introducing into a cell a vector comprising a EPC cell-specific promoter coupled to at least one selectable marker gene; expressing the selectable marker gene from the cell specific promoter in the cell; and detecting expression of the marker gene in the cell, thereby identifying the EPC, wherein said EPC do not express at least one or more of MHC class I, MHC class II, CD44, CD45, CD13, CD34, CD49c, CD66b, CD73, CD105, and CD90 cell surface proteins; and said EPC expresses at least one or more of Oct-4, Nanog, Sox-2, Rex-1, GDF-3, Stella, FoxD3, or Polycomb embryonic transcription factors.

In some embodiments, the EPC cell-specific promoter is a VEGF promoter, a VEGF-receptorin some embodiments, the EPC specific promoter is flanked by loxP sites.

In some embodiments, the vector is a retroviral vector. In some embodiments, the selectable marker gene encodes a fluorescent protein, such as but limited to Green Fluorescent Protein (GFP). In some embodiments, the vector comprises two selectable marker genes, the two selectable marker genes comprise a fluorescent protein, a protein sensitive to drug selection, a cell surface protein or any combination thereof. Further aspects of the disclosure relate to a method of generating a EPC comprising the steps of introducing into a population of EPC a vector comprising a promoter, such as a regenerative cell-specific promoter, coupled to at least one selectable marker gene, wherein said EPC do not express MHC class I, MHC class II, CD44, CD45, CD13, CD34, CD49c, CD73, CD105 and CD90 cell surface proteins; expressing the selectable marker gene from the EPC specific promoter in said cell population; and detecting expression of the marker gene in the EPC. In some embodiments, the methods further comprise a step of transfecting the regenerative EPC cells with OCT-4 transcription factor, thereby enhancing the regenerative activity of the EPC. In some embodiments, the methods further comprise a step of fusing the EPC with cells having a pluripotent ability thereby generating EPCs with enhanced regenerative activity.

Culture conditioned media may be concentrated by filtering/desalting by means known in the art. In one embodiment Amicon filters, or substantially equivalent means, with specific molecular weight cut-offs are utilized, said cut-offs may select for molecular weights higher than 1 kDa to 50 kDa.

The cell culture supernatant may alternatively be concentrated using means known in the art such as solid phase extraction using C18 cartridges (Mini-Spe-ed C18-14%, S.P.E. Limited, Concord ON). Said cartridges are prepared by washing with methanol followed by deionized-distilled water. Up to 100 ml of stem cell or progenitor cell supernatant may be passed through each of these specific cartridges before elution, it is understood of one of skill in the art that larger cartridges may be used. After washing the cartridges material adsorbed is eluted with 3 ml methanol, evaporated under a stream of nitrogen, redissolved in a small volume of methanol, and stored at 4.degree. C.

Before testing the eluate for activity in vitro, the methanol is evaporated under nitrogen and replaced by culture medium. Said C18 cartridges are used to adsorb small hydrophobic molecules from the stem or progenitor cell culture supernatant, and allows for the elimination of salts and other polar contaminants. It may, however be desired to use other adsorption means in order to purify certain compounds from said EPC supernatant. Said EPC concentrated supernatant may be assessed directly for biological activities useful for the practice of this invention, or may be further purified. In one embodiment, said supernatant of EPC culture is assessed for ability to stimulate proteoglycan synthesis using an in vitro bioassay. Said in vitro bioassay allows for quantification and knowledge of which molecular weight fraction of supernatant possesses biological activity. Bioassays for testing ability to stimulate proteoglycan synthesis are known in the art. Production of various proteoglycans can be assessed by analysis of protein content using techniques including mass spectrometry, column chromatography, immune based assays such as enzyme linked immunosorbent assay (ELISA), immunohistochemistry, and flow cytometry.

Further purification may be performed using, for example, gel filtration using a Bio-Gel P-2 column with a nominal exclusion limit of 1800 Da (Bio-Rad, Richmond Calif.). Said column may be washed and pre-swelled in 20 mM Tris-HCl buffer, pH 7.2 (Sigma) and degassed by gentle swirling under vacuum. Bio-Gel P-2 material be packed into a 1.5.times.54 cm glass column and equilibrated with 3 column volumes of the same buffer. Amniotic fluid stem cell supernatant concentrates extracted by C18 cartridge may be dissolved in 0.5 ml of 20 mM Tris buffer, pH 7.2 and run through the column. Fractions may be collected from the column and analyzed for biological activity. Other purification, fractionation, and identification means are known to one skilled in the art and include anionic exchange chromatography, gas chromatography, high performance liquid chromatography, nuclear magnetic resonance, and mass spectrometry. Administration of supernatant active fractions may be performed locally or systemically.

In some embodiments of the invention the use of other TLR-4 antagonists are utilized to activate EPC said TLR4 antagonists is disclosed, possible TLR-4 antagonists for use in the invention include: LPS and lipid A from Rhodobacter sphaeroides; LOS from Bartonella Quintana; LPS from Oscillatoria Planktothrix FP1; curcumin from Curcuma longa, sulforaphane and iberin from cruciferous vegetables [1-7]; xanthohumol from hops and beer [8-14], and celastrol from Tripterygium wilfordii [15-17].

In one embodiment of the invention, EPCs are cultured in a manner to allow viability. Numerous culture techniques are known in the art. Tissue culture solutions (media) that allow for stem cell viability include Roswell Park Memorial Institute (RPMI-1640), Dublecco's Modified Essential Media (DMEM), Eagle's Modified Essential Media (EMEM), Optimem, and Iscove's Media. Said media is usually supplemented with a source of serum, or alternatively serum-free media may be used. Serum from fetal calves is typically used at a concentration ranging from 2%-20%, more preferably at approximately 10%. In some embodiments, said fetal calf serum is heat-inactivated by incubation at 55 Celsius for 1 hour in order to neutralize complement activity. In other embodiments human serum is used as a substitute for fetal calf serum. Conditioned media is collected from cells that are originally plated at a concentration between 20-8000 cells/cm(2), more preferably between 2000-8000 cells/cm(2), and more preferably at an approximate concentration of 4000 cells/cm(2). One of skill in the art may with minimal experimentation identify ideal concentration of cells to be cultured based on assessment of viability, growth factor production, and generation of anti-inflammatory. Conditioned media may be collected at 24-72 hours of culture, filtered to remove cellular debris and depending on the concentration of regenerative compounds desired, may be concentrated. Means of concentration are known to one of skill in the art. For example, molecular weight filter such as an Amicon 3000 Stir Cell can be used to reduce the volume and at the same time remove low molecular weight salts. Alternatively, concentration of EPC produced regenerative factors of the conditioned media may be achieved by means of column chromatography; or, lyophilization to remove the water in the medium, effectively concentrating the effective components.

In some embodiments EPC conditioned media is utilized as part of a formulation with other therapeutic compounds whereas said formulation is administered intradiscally or systemically in order to induce proteoglycan production from the disc. compounds can be vitamin A, vitamin C, vitamin D, vitamin E, vitamin K, folic acid, choline, vitamin B1, vitamin B2, vitamin B5, vitamin B6, vitamin B12, biotin, nicotinamide, betacarotene, coenzyme Q, selenium, superoxide dismutase, glutathione peroxide, uridine, creatine succinate, pyruvate, dihydroxyacetone), acetyl-L-carnitine, alpha-lipoic acid, cardiolipin, omega fatty acid, lithium carbonate, lithium citrate, calcium, or any combination thereof. In some aspects, the compounds are anti-inflammatory agents. In some aspects, the anti-inflammatory agents are the anti-inflammatory agent is Alclofenac; Alclometasone Dipropionate; Algestone Acetonide; Alpha Amylase; Alpha-lipoic acid; Alpha tocopherol; Amcinafal; Amcinafide; Amfenac Sodium; Amiprilose Hydrochloride; Anakinra; Anirolac; Anitrazafen; Apazone; Ascorbic Acid; Balsalazide Disodium; Bendazac; Benoxaprofen; Benzydamine Hydrochloride; Bromelains; Broperamole; Budesonide; Carprofen; Chlorogenic acid; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Ellagic acid; Endry sone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Glutathione; Halcinonide; Halobetasol Propionate; Halopredone Acetate; Hesperedin; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Lycopene; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Oleuropein; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Pycnogenol; Polyphenols; Prednazate; Prifelone; Prodolic Acid; Proquazone; Proxazole; Proxazole Citrate; Quercetin; Reseveratrol; Rimexolone; Romazarit; Rosmarinic acid; Rutin; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrahydrocurcumin; Tetrydamine; Tiopinac; Tixocortol Pivalate; Tolmetin; Tolmetin Sodium; Triclonide; Triflumidate; Zidometacin; Zomepirac Sodium. In some aspects the compounds are bioactive compounds including but not limited to growth factors, cytokines, antibodies, antibody fragments, and/or organic molecules of a mass of less than 5000 daltons. The compounds may be administered concurrently with a composition of the current disclosure. Alternatively, the compounds may be administered before and/or after the composition is administered to a subject.

### REFERENCES

ADDIN EN.REFLIST 1. Youn, H. S., et al., Sulforaphane suppresses oligomerization of TLR4 in a thiol-dependent manner. J Immunol, 2010. 184(1): p. 411-9.
2. Shan, Y., et al., Sulphoraphane inhibited the expressions of intercellular adhesion molecule-1 and vascular cell adhesion molecule-1 through MyD88-dependent toll-like receptor-4 pathway in cultured endothelial cells. Nutr Metab Cardiovasc Dis, 2012. 22(3): p. 215-22.
3. Zhao, L., J. Y. Lee, and D. H. Hwang, Inhibition of pattern recognition receptor-mediated inflammation by bioactive phytochemicals. Nutr Rev, 2011. 69(6): p. 310-20.
4. Kim, S. Y., et al., PI3K/Akt contributes to increased expression of Toll-like receptor 4 in macrophages exposed to hypoxic stress. Biochem Biophys Res Commun, 2012. 419(3): p. 466-71.
Koo, J. E., et al., Sulforaphane inhibits the engagement of LPS with TLR4/MD2 complex by preferential binding to Cys133 in MD2. Biochem Biophys Res Commun, 2013. 434(3): p. 600-5.
6. Folkard, D. L., et al., Suppression of LPS-induced transcription and cytokine secretion by the dietary isothiocyanate sulforaphane. Mol Nutr Food Res, 2014. 58(12): p. 2286-96.
7. Qu, X., et al., Sulforaphane epigenetically regulates innate immune responses of porcine monocyte-derived dendritic cells induced with lipopolysaccharide. PLoS One, 2015. 10(3): p. e0121574.
8. Cho, Y. C., et al., Differential anti-inflammatory pathway by xanthohumol in IFN-gamma and LPS-activated macrophages. Int Immunopharmacol, 2008. 8(4): p. 567-73.
9. Peluso, M. R., et al., Xanthohumol and related prenylated flavonoids inhibit inflammatory cytokine production in LPS-activated THP-1 monocytes: structure-activity relationships and in silico binding to myeloid differentiation protein-2 (MD-2). Planta Med, 2010. 76(14): p. 1536-43.
Fu, W., et al., Determination of the binding mode for anti-inflammatory natural product xanthohumol with myeloid differentiation protein 2. Drug Des Devel Ther, 2016. 10: p. 455-63.
11. Chen, G., et al., Discovery of new MD2-targeted anti-inflammatory compounds for the treatment of sepsis and acute lung injury. Eur J Med Chem, 2017. 139: p. 726-740.
12. Hege, M., et al., An iso-alpha-acid-rich extract from hops (Humulus lupulus) attenuates acute alcohol-induced liver steatosis in mice. Nutrition, 2018. 45: p. 68-75.
13. Li, F., et al., Xanthohumol attenuates cisplatin-induced nephrotoxicity through inhibiting NF-kappaB and activating Nrf2 signaling pathways. Int Immunopharmacol, 2018. 61: p. 277-282.
14. Chen, C. Y., C. L. Kao, and C. M. Liu, The Cancer Prevention, Anti-Inflammatory and Anti-Oxidation of Bioactive Phytochemicals Targeting the TLR4 Signaling Pathway. Int J Mol Sci, 2018. 19(9).
15. Ni, H., et al., Celastrol inhibits lipopolysaccharide-induced angiogenesis by suppressing TLR4-triggered nuclear factor-kappa B activation. Acta Haematol, 2014. 131(2): p. 102-11.
16. Lee, J. Y., et al., Celastrol blocks binding of lipopolysaccharides to a Toll-like receptor4/myeloid differentiation factor2 complex in a thiol-dependent manner. J Ethnopharmacol, 2015. 172: p. 254-60.
17. Kang, S. W., et al., Celastrol attenuates adipokine resistin-associated matrix interaction and migration of vascular smooth muscle cells. J Cell Biochem, 2013. 114(2): p. 398-408.

1. A method of creating an endothelial progenitor cell (EPC) conditioned media comprising the steps of: a) obtaining a cellular population; b) dedifferentiating said cellular population; c) inducing differentiation of said dedifferentiated cell into EPC; and d) culturing said EPC in a liquid media to obtain a conditioned media.

2. The method of claim 1, wherein said cellular population is an EPC.

3. The method of claim 2, wherein said EPC is derived from a source selected from the group consisting of: a) peripheral blood; b) bone marrow; c) placenta; d) cord blood; e) menstrual blood; f) amniotic fluid; and g) cerebral spinal fluid.

4. The method of claim 2, wherein said EPC express VEGF-receptor 2.

5. The method of claim 2, wherein said EPC express interleukin-3 receptor.

6. The method of claim 1, wherein said cellular population is monocytes.

7. The method of claim 1, wherein said cellular population is hematopoietic stem cells.

8. The method of claim 1, wherein said cellular population is a mesenchymal stem cell.

9. The method of claim 8, wherein said naturally occurring mesenchymal stem cells are tissue derived.

10. The method of claim 8, wherein said naturally occurring mesenchymal stem cells are derived from a bodily fluid.

11. The method of claim 10, wherein said tissue derived mesenchymal stem cells are derived from umbilical cord tissue.

12. The method of claim 10, wherein said tissue derived mesenchymal stem cells are isolated from tissues containing cells selected from the group consisting of: endothelial cells, epithelial cells, dermal cells, endodermal cells, mesodermal cells, stems, osteocytes, chondrocytes, natural killer cells, dendritic cells, hepatic cells, pancreatic cells, stromal cells, salivary gland mucous cells, and salivary gland serous cells.

13. The method of claim 1, wherein said dedifferentiation is accomplished by introduction into cells proteins capable of inducing dedifferentiation.

14. The method of claim 13, wherein said pluripotency marker is TRA-1-60.

15. The method of claim 13, wherein said proteins capable of inducing dedifferentiation are selected from the group consisting of: a) OCT4; b) NANOG; c) KLF-1; d) SOX-2; and e) h-RAS.

16. The method of claim 13, wherein mRNA is introduced into said cells in order to induce expression of pluripotency inducing genes.

17. The method of claim 1, wherein said dedifferentiated cells are capable of proliferating for more than 50 passages.

18. The method of claim 1, wherein said redifferentiated cells are capable of proliferating for more than 50 passages and have stable karyotype.

19. The method in claim 1, wherein said redifferentiated cells are a direct therapeutic effector.

20. The method in claim 1, wherein said redifferentiated cells are more potent than naturally occurring EPCs based on a) VEGF production b) nitric oxide release and c) angiogenesis/capillary formation assay.
